(19) United States
(12) Patent Application Publication
(10) Pub. No.: US 2002/0103139 A1
(43) Pub. Date: Aug. 1, 2002

(54) Solid self-emulsifying controlled release drug delivery system composition for enhanced delivery of water insoluble phytosterols and other hydrophobic natural compounds for body weight and cholestrol level control

(76) Inventors: M. Weisspapir, North York (CA); J. Schwarz, North York (CA)

Correspondence Address: Paul Sharpe, Marks & Clerk, P.O. Box 957, Station B, Ottawa, ON, K1P 5S7, CA

(21) Appl. No.: 09/726,526

(22) Filed: Dec. 1, 2000

(51) Int. Cl. A61K 31/56; A61K 31/704

(52) U.S. Cl. 514/26; 514/178

(57) ABSTRACT

There is provided a composition for body weight and cholesterol control as well as a process for preparation of the composition. In one embodiment the composition includes at least one phytosterol in an amount between 5% and 50% by weight. The composition further includes a physiologically or pharmaceutically acceptable surfactant in an amount between 0.1% and 50% by weight and a lipid phase for containing the phytosterol and the surfactant, the lipid phase present in an amount from 1% and 50% by weight. A pharmaceutically acceptable excipient for facilitating absorption of the lipid phase is also present in an amount from between 10% and 80% by weight.

### FIELD OF THE INVENTION

The present invention relates to chemical formulations for regulating excessive bodyweight of human patients and for normalizing plasma cholesterol levels.

### BACKGROUND OF THE INVENTION

Every generation has had variations of medicines for treating excessive weight and obesity. In the 1930s orange pekoe tea and bath salts were employed. Then 1950s brought laxatives and minerals, and in the 1960s and 1970s amphetamines were widely used. In 1996, the FDA approved a new anti-obesity drug dexfenfluramine (Redux®), that induced the escalation of serotonin production. Serotonin possesses a pronounced antiappetite activity. Dexfenfluramine along with phentermine and/or fenfluramine (other appetite suppressants, so called “Phen-Fen” combination) is still available on the market.

These drugs have undesirable side effects such as: nervousness, palpitations, insomnia, constipation, inter alia. Dexfenfluramine has been known to potentially cause incurable diseases including—primary pulmonary hypertension and heart valve injury. Moreover, as demonstrated in scientific review, the patients treated by dexfenfluramine and fenfluramine only lost an average of 5 pounds more than that attainable with placebo.

Many medicines of natural origin (plant, animal or mineral) have been mentioned as suitable for weight control in humans. The list of active ingredients used for this purpose contains thousands of substances. Some are efficacious, but many show negligible activity or a complete lack of performance with some showing serious side effects.

The best known medicines for weight loss used as additives to diet with decreased levels of fat, carbohydrates and increased content of vitamins. Such remedies may comprise natural fibers (chitin, glucomannan, bran, microcrystalline cellulose, hemicellulose, lignin, psyllium husk, kelp), gums (tragacanth, guar gum carrageenan, glucomannanes and alginates, etc.), which swell in the stomach and create feeling of fullness. Fibers can absorb cholesterol thus improving cardiovascular conditions of the patient.

Stimulators of the central nervous system and related compounds (ephedrine, amphetamines, fenfluramine, phentermine, xantines) work predominantly as appetite suppressants. Unfortunately, these compounds demonstrate side effects, including increased blood pressure, asthmatic spasms, heart valve degeneration, arrhythmia, and insomnia and can be addictive. Natural stimulants used as appetite suppressants examples of which include Panax Ginseng, Cola nuts, Eleuterococcus senticosus, Aralia manshurica, Schizandra chinensis, have been known to induce blood pressure elevation.

Metal supplements (Cu, Cr, V, Mo, Zn, etc.) can regulate metabolism of carbohydrates, fats and proteins. Many of the metals are inherent in different enzyme molecules. Chromium, for example, is part of glucose tolerance factor (GTF) and regulates glucose penetration into cells.

In terms of vitamins and related compounds, one of the most prominent roles is antioxidant activity and free radical scavenging essential for fat utilization. Fat burning creates metabolic waste products, which include ketons and lipid peroxides. Ascorbic acid, beta-carotene, vitamins A and E can protect tissues from damage induced by free radicals.

Lipogenesis inhibitors such as ((+)Hydroxycitric acid—Garcinia cambojia) decrease fat synthesis in the body. During the normal metabolism of foods, carbohydrates which are neither used immediately for energy nor stored as glycogen, are converted into fats in the liver by the enzyme ATP citrate-lyase. (+)Hydroxycitric acid inhibits this enzyme and conjugated coenzyme A thus inhibiting transfer of carbohydrates to fat and formation of the low density lipoproteins (LDL) and triglycerides.

Thyroid activator (L-Tyrosine, Thyroxine Iodine). Thyroid gland activity connected with Na/K ATPase enzymes influence the rate at which fat is burned for energy.

The non-essential amino acid, L-Tyrosine, converts to norepiniphrine which, in turn, gives a sense of fullness. The importance of tyrosine is based on the fact that it is a direct precursor to thyroxine (Thyroid hormone) as well as being a precursor to adrenaline and noradrenaline. Thyroxine deficiency results in a series of conditions including excess weight gain, decreased basal metabolism, etc.

Iodine occurs primarily in seafood and seaweeds. Iodine is necessary for normal physical and mental growth & development. Iodine is an essential component of thyroid hormones, aids in the development and functioning of the thyroid gland, regulates the body's production of energy, and helps burn excess fat by stimulating the rate of metabolism. Iodine deficiency causes goiter, and may result in an enlarged thyroid gland, slow mental reaction, dry skin & hair, weight gain, and/or loss of physical & mental vigor.

Regarding fat absorbers (Phytosterols, Sitostanol), vegetable-derived phytosterols mainly include sitosterol (and its glucoside—sitosterolin), campesterol, sitostanol and stigmasterol. Sterols are essential components to both animal and plant organisms.

Sitosterol promotes the conversion of linoleic acid to polyunsaturated fatty acid that is necessary for synthesis of prostaglandins and leukotrienes. Furthermore sitosterol can be used as a resource for synthesis of steroid hormones. Chemically, sitosterol is an analogue of cholesterol and interferes with cholesterol absorption in GIT that leads to reductions in blood cholesterol. Phytosterols show poor absorption and their levels in serum 20 are very low. Studies have shown that a relatively small amount of sitostanol (1.5 g/day, 5-α-reduced metabolite of sitosterol), in contrast with high doses of sitosterol (20 g/d), are needed to lower serum cholesterol level, particularly when a soluble form of sterol is used. Reduced cholesterol absorption has been found to promote the fecal excretion of both dietary and endogenous biliary cholesterol, as was established in U.S. Pat. No. 5,965,449, issued at Novak et al.

The most effective formulation regarding reduction in cholesterol absorption is micellar sitostanol. This special form of sitostanol, that passed through 5 μm filter, allowing direct delivery into the intestinal micellar phase and reduces cholesterol absorption by up to 37%. No significant difference was observed between cholesterol reduction by doses of 700 and 300 mg of sitostanol. Accordingly, the form of the phytosterols presented in the small intestine is the prime determination factor of its efficacy.

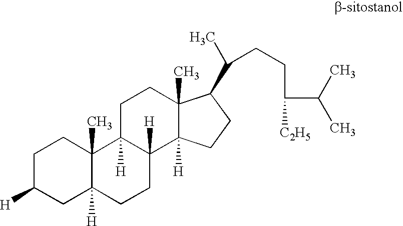

In patent application WO 99/63841, different approaches to increase phytosyterol bioavailability by improvement of solubility and dispersability were employed: including incorporation in emulsions, micellar solutions, microemulsions, solid dispersions, liposomes, bile acid salts systems and cyclodextrin complexes. These compositions were incorporated into food, such as chocolate, margarine, bread, cereal, spreads, etc. They demonstrated high efficacy, but the total amount of additional components used for preparation of such compositions are bulky as established in U.S. Pat. No. 5,747,464, issued to Jackie Ray See.

With respect to the medium chain triglycerides, (MCT), these are not stored as body fat and do not need bile for digestion. They are consumed in mitochondria of the cells to provide energy. MCTs do seem to promote lipolysis (fat burning) and they have a pronounced thermogenic effect. Nevertheless, subjects consuming MCT lost weight, but in the same rate as a similar low calorie diet. However, MCTs do serve to protect the body's protein in the lean tissues in low calorie and low carbohydrate diets.

The appetite suppressants (L-Phenylalanine, L-Tyrosine, blue-green algae, Synephrine—Citrus aurantium).

L-Phenylalanine is an essential amino acid and through a series of biochemical reactions, it is easily converted to L-tyrosine. L-tyrosine is a precursor to a number of neurotransmitters and hormones, such as adrenaline, dopamine and thyroid hormones. These are important regulators of the metabolism and nervous system. L-tyrosine is also a primary precursor to hypothalamic hormone cholecystokinin (CCK) that plays an important role in appetite regulation.

Blue-green algae (Spirulina) contains considerable amounts of complete protein, essential fatty acid, vitamins and minerals but low in calories. It is known experimentally that concentrated complete foods tend to reduce appetite and partition energy into lean tissues.

Citrus aurantium (Bitter orange) contains alkaloid synephrine that close to ephedrine, but has few of ephedrine's side effects because synephrine does not easily cross the blood-brain barrier. Synephrine stimulates receptors on the membrane of fat cells, which activate fat-burning enzymes. Synephrine suppresses appetite, increases the rates of lipolysis and metabolism.

Coenzyme Q10 (CoQ10, also known as ubiquinone because it is present in nearly all cells), is obtained from the diet (mainly from fatty fish, internal organs, and whole grains) and is also produced by the body. Some have suggested that CoQ10 will soon be classified as an oil-soluble vitamin because it is now known to be essential and because its deficiency leads to healthy problems. Chemically, its structure is related to that of the vitamins E and K. The amino acid methionine is essential for its production. CoQ10 appears to be virtually non-toxic.

CoQ10 action is similar to L-carnitine. It plays an important role with mitochondria, the intracellular organelles which produce energy. The important energy storage chemical ATP (adenosine triphosphate) is produced by the mitochondria with the aid of CoQ10. The coenzyme is also a potent antioxidant, as is suggested by its structural similarity to vitamin E. It quenches free radicals by donating its own electrons and also prevents lipid peroxidation in the body as do other antioxidants.

As with L-carnitine, CoQ10 may demonstrate benefit to those who are overweight because it improves the efficiency of energy production at the cellular level. There is some evidence indicated that certain inefficiencies are related to genetic inheritances concerning the body's ability to manufacture this coenzyme. About half of those with a family history of obesity do not respond to meals as they should. A normal response to a meal is for the body to slightly raise its rate of energy production. Many of those who are overweight do not have this response. Blood serum tests for levels of CoQ10 indicate that these levels in almost 50% of the obese subjects were in fact deficient. (P. W. Singal et al., “on the role of coenzyme Q10 in cardiovascular diseases”, Cardiovascular Research, 43 (1999), pp. 250-251).

A serious obstacle for CoQ10 is its low water solubility and associated low bioavailability of CoQ10 in traditional oral delivery forms such as tablets and capsules. Attempts to improve bioavailability by mixing CoQ10 with oil or oil and lecithin showed inconsequential response. Recently, an improved formulation, based on self-emulsifying vehicle, was announced (R. K. Chpra et al., “Bioavailability of Coenzyme Q10 in humans”, Int.J.Vit. Nutr. Res). This technology (soft gelatine capsules, filled with self-emulsifying oil based mixture) is suitable only for immediate release formulations.

As for lipotropic factors, the fat mobilizing nutrients (Vitamin B5 and B6, Methionine, Guggulipid—Commiphora mukul, Synephrine—Citrus aurantium, Betaine HCl, Choline, Inositol, Dandelion, Barberry, Bearberry, Oregon grape, Milk thistle etc.), the primary site of lipotropic action is the liver. Lipotropics prevent the accumulation of fat in liver and usually aid in the detoxification of metabolic wastes and other toxins.

The gum resin extract from the bark of Commiphora mukul (gugul) contains a combination of steroids, diterpenoids, alipathic esters, and carbohydrates. Of considerable pharmacological interests are the unique steroids found in Commiphora mukul, the guggulsterones. Several different guggulsterones have been identified: guggulsterols I, II, III, IV, and V; E-guggulsterone; and Z-guggulsterone.

The hypolipidemic action of guggul was first reported in 1966. Experiments showed that guggul lowered the serum cholesterol levels and body weight of hypercholesterolemic rabbits and protected the animals against hypercholesterolemia and atherosclerosis induced by an atherogenic diet.

Both guggul and its purified extracts have proven to be effective hypolipidemic agents in patients with ischemic heart disease, hypercholesterolemia, obesity, and hyperlipidemia. Fifty patients with symptoms of ischemic heart disease treated with an oral daily dose of 10-15 grams of guggul for three months experienced a 25% decrease in total cholesterol and a 30% reduction in triglycerides, representing a significant change compared to controls. Twenty-two patients with primary hyperlipidemia were given a 1,500 mg daily dose of gugulipid for six weeks. Serum cholesterol levels were significantly reduced in 59% of the patients, evident at two weeks after initiation of treatment. Among responders, serum cholesterol and triglyceride levels were lowered 24.5% and 27.3%, respectively. The average reduction in serum cholesterol and triglycerides in response to treatment with the standardized extract was 11% and 16.8%, respectively. HDL-cholesterol was increased in 60% of the patients who responded to treatment with the C. mukul extract.

Guggul has been shown to be a useful adjunct to dietary modification in hypercholesterolemic patients.

The hypolipidemic action of guggul is attributed to the presence of guggulsterones, and at least four mechanisms have been proposed to explain their activity. First, guggulsterones might interfere with the formation of lipoproteins by inhibiting the biosynthesis of cholesterol in the liver. Second, guggulsterones have been shown to enhance the uptake of LDL by the liver through stimulation of the LDL receptor binding activity in the membranes of hepatic cells. Third, guggulsterones increase the fecal excretion of bile acids and cholesterol resulting in a low rate of absorption of fat and cholesterol in the intestines. Finally, guggulsterones directly stimulate the thyroid gland. Because serum lipids, including cholesterol, are reduced in response to increased levels of circulating thyroid hormones, the effect of guggulsterones on the thyroid gland might explain the hypolipidemic activity and weight loss property of guggul.

DHEA (dehydroepiandrosterone) is the most common steroid present in our bodies, after cholesterol. Produced by our adrenal glands and gonads, it is a precursor for the body's manufacture of many other significant hormones. The importance of DHEA is highlighted by findings that approximately 50% of total androgens in adult men are derived from DHEA. And in women, the formation of estrogens in peripheral tissues from DHEA/DHEAS is approximately 75% before menopause, and close to 100% after menopause.

DHEA inhibits an enzyme glucose-6-phosphate dehydrogenase which is responsible for storing fat. DHEA increases cell sensitivity to insulin and to thyroid hormone that promote thermogenesis, fat metabolism and energy production.

Pure aminoacids (L-Carnitine) increase the transport of fatty acids across the cellular membrane and into the mitochondria, where they are burned. L-Ornithine, L-Arginine, L-Tyrosine, L-Tryptophan and L-Glycine stimulate the pituitary gland's secretion of somatotrapin—Growth Hormone (GH). GH stimulates protein production and increases muscle tissue growth. GH generates release fatty acids and contributes to their utilization.

Fatty acids (Gamma-Linolenic acid, Conjugated Linoleic acid) increase the level of Na/K ATPase in brown fat. GLA modifies the mode that fats are broken down and stored. GLA increases the ratio of saturated fats to monosaturated fats in different tissues. This effect led to a reduction in food consumption, a decrease in fat storage, an improvement of HDL/LDL proportion and total cholesterol diminution.

### SUMMARY OF THE INVENTION

It has been found that a self-emulsifying composition, loaded with selected biologically active components, is useful for delivery of these components as a submicron emulsion. Such an emulsion is either readily absorbed in the gastro-intestine, thus providing significant improvement in bioavailability or depending on the substance properties, actively interacts with such meal components as cholesterol and with cholesterol sensitive receptors, inhibiting penetration of cholesterol into the body. It allows significantly improved efficacy of included ingredients in obesity treatment and cholesterol level control.

One object of one embodiment of the present invention is to provide a convenient easy-to-swallow formulation with sufficient loading of the (poor water soluble) components, having poor inter solubility where the named components are dissolved, suspended or dispersed in the self-emulsifying lipid blend, and providing continuous release of the included active components from the composition as the oil-in-water emulsion is gradually released from the composition. To achieve such performance, the self-emulsifying blend is mixed into a tablet forming composition which may comprise lipids, surfactants, fillers, polymers and other excipients to form a stable formulation.

A further object of one embodiment of the present invention is to provide a composition for bodyweight and cholesterol control, comprising: at least one phytosterol in an amount between 5% and 50% by weight; a physiologically or pharmaceutically acceptable surfactant in an amount between 0.1% and 50% by weight; a lipid phase for containing the phytosterol and the surfactant, the lipid phase present in an amount from 1% and 50% by weight; and a pharmaceutically acceptable excipient for facilitating absorption of the lipid phase present in an amount from between 10% and 80% by weight.

A physiological acceptable nutrient may be added to the mixture in an amount of between 0.005% and 90% by weight. Suitable nutrients include vitamin compounds, coenzymes, aminoacids, and minerals.

Active components possessing different types of fat and cholesterol regulating activities, can be selected from those mentioned supra. In view of the fact that most problematic in efficacy are hydrophobic, water insoluble compounds, they are materials of choice for inclusion in self-emulsifying compositions. For example, the main problem for sitosterol formulations is extremely low solubility of such sterols in the body fluids. This is why usual doses of phytosterols are quite high, and efficacy is inconsistent. With the inclusion of hydrophobic water insoluble molecules into the self-emulsifying base with subsequent formation of submicron particles of finely dispersed oil phase with dissolved components, a tremendous improvement in absorption in the gastrointestinal tract is observed. Such components as phytosterols (sitosterol, sitostanol, campesterol); triterpene compounds (gugglul), saw palmetto extract (fatty acids), polycosanol (octacosanol), coenzyme Q10, and many others can be successfully used in the formulation. Water soluble components can also efficiently be included into preparation thus increasing formulation efficacy and potential.

A still further object of one embodiment of the present invention is to provide a composition for bodyweight and cholesterol control, comprising: a self-emulsifying base providing in the body submicron particles of dissolved components, the components including: at least one phytosterol in an amount between 5% and 50% by weight; a physiologically or pharmaceutically acceptable surfactant in an amount between 0.1% and 50% by weight; a lipid phase for containing the phytosterol and the surfactant, the lipid phase present in an amount from 1% and 50% by weight; and a pharmaceutically acceptable excipient for facilitating absorption of the lipid phase present in an amount from between 10% and 80% by weight.

The self-emulsifying lipid microemulsion may comprise of food grade oils and fats (soya oil, olive oil, kernel oil, cocoa butter etc.), pharmaceutically acceptable glycerides and glycerin fatty acid esters (MCT, tricaprin, trimyristin, triolein and many others), mono- and diglycerides, their mixtures and derivatives (Capful, Miglyol, Myvacet, Witepsol etc.) fatty and aliphatic acid and fatty acids esters (oleic and linoleic acid, ethyl oleate, isopropylmyristate, isopropylpalmitate, isostearic esters, etc.), pharmaceutical plasticizers (triethylcitrate, diethyladipate, diethylsebacate, ethyltributylcitrate, dioctylphtalate), lipidic pharmaceutical compounds (tocopherols and tocopherol esters, retinol acetate and palmitate, cholesteryl derivatives), phosphatidylcholine derivatives (soy and egg lecithin and analogs), and other compounds, selected to obtain desirable solubility of the loaded drug.

Yet another object of one embodiment of the present invention is to provide a method of preparing a composition as claimed in claim 1, comprising the steps of:

a) dissolving the at least one phytosterol and the surfactant in the lipid phase;

b) formulating a self-emulsifying mixture by mixing dissolved materials from step a) with a volatile organic solvent and granulation of the obtained solution with nutrient and other excipients;

c) drying the mixture from step b);

d) mixing dried mixture from step c) with other granulating components; and

e) granulating the mixture from step d).

To achieve the self-emulsifying behavior of the drug loaded composition, different surfactant or surfactant mixtures with the HLB value, adapted to an oil and drug type, may be added in an appropriate ratio. The surfactant may be selected from non-ionic groups such as polyoxyethylated fatty acids (PEG-stearates, PED-laurates, Brij®), PEG-ethers (Mirj®), sorbitan derivatives (Tweens) aromatic compounds (Tritons®), PEG-glycerides (PECEOL™), PEG-PPG copolymers (Pluronics, Poloxamers, Jeffamines), Polyglycerines, PEG-tocopherols, propylene glycol derivatives, sugar and polysaccharide alkyl and acyl derivatives (octylsucrose, sucrose stearate, laurolydextran etc.). Other types of surfactants, such as anionic (soaps, sulfonates), cationic (CTAB), zwitterionic and amphoteric, are also suitable.

The HLB value of the surfactant mainly depends on the lipidic composition and to a lesser extent on the drug structure. Lipidic additives, such as fatty acid glycerides, are often used as release retardants for hydrophilic matrices. The hydrophobic nature decreases water penetration rate and hydration of the polar polymer, and reduces dissolution rate. Another important feature is the lubricant properties of the hard glycerides and fatty acids.

Surfactants, mainly non-ionic, are also widely suitable in extended release formulations for poorly soluble drugs. They help to keep the drug in dissolved or dispersed state and prevent its precipitation by dissolution in forming micellar solution after contact with water. They are added in relatively large amounts.

The type and level of excipients added to self-emulsifying composition is also important. Improper choice of these components leads to weak tablet formation or makes tablet preparation impossible; the emulsification process and controlled release pattern may also be affected. Accurate selection of these components is based on consideration of all component properties separately and in combination.

### DETAILED DESCRIPTION OF THE PREFERRED EMBODIMENTS

Reference will now be made to several examples to further elucidate various embodiments of the invention.

### EXAMPLE 1

### Self-Emulsifying Composition Containing Water Insoluable Beta-Sitosterol, Octacosanol, Saw Palmetto Extract, Alpha-Lipoic Acid, Tocopherol Acetate and Coenzyme Q10 in Combination with Water Soluble Herbal Extracts, Vitamins and Trace Metals

As a first example of the first formulation, the slowly dissolving composition contained 20 mg of beta-sitosterol, 1 mg of octacosanol, extracted from sugar cane, 40 mg of saw palmetto liquid extract, 20 mg of Coenzyme Q10 (Ubiquinone) and 20 mg of alpha-lipoic (thiooctic) acid per tablet. The oil phase comprised alpha-tocopherol acetate (vitamin E acetate). PEG-40 stearate (Mirj® 52) was used as surfactant with optimal HLB value for effective emulsification of the oil phase.

To obtain the solid self-emulsifying composition, active ingredients and surfactants were mixed with lipid components, heated and dissolved using suitable stirrer. Ethyl alcohol was added to the obtained mixture and this liquid was used as a granulation solution for granulation with the remaining compounds of part A (table 1), using a low speed planetary mixer (Hobart™). The granulate was dried at 40° C., passed through a stainless sieve (16 mesh), subsequently mixed with other components (part B, table 1) and granulated with ethyl alcohol in the same equipment. After drying, the granulate was passed through the stainless sieve (16 mesh), mixed with lubricant (magnesium stearate) and compressed into oval shape tablets using a single punch tablet press having a pressing force 1.5-4 tons. The resulting tablet had a hardness of 3-6 kp.

Additionally, preferred granulation can be filled into gelatin capsules to obtain another traditional solid oral delivery form.

TABLE 1

| | COMPOUND | PER TABLET, mg |
|---|---|---|
| | Part A | |
| 1 | Beta-sitosterol | 20 |
| 2 | Octacosanol | 1 |
| 3 | Saw palmetto 90% liquid extract | 40 |
| 4 | Coenzyme Q10 | 10 |
| 5 | Alpha-lipoic acid | 20 |
| 6 | Ethyl alcohol 95% USP for granulation | 40 |
| 7 | Scullcap chinensis 5:1 PE | 20 |
| 8 | Alpha-Tocopherol acetate | 50 |
| 9 | Rutin | 40 |
| 10 | Chromium polinicotinate | 0.2 |
| 11 | Folic acid | 0.5 |
| 12 | Forscolin (as 20% dry extract) | 40 |
| 13 | Citrus Aurantium PE (8% synephrine) | 50 |
| 14 | TPGS | 25 |
| 15 | Mirj ® 52 | 25 |
| | Part B | |
| 16 | Fumed silica (Cab-O-Sil) | 60 |
| 17 | Dibasic Calcium Phosphate | 60 |
| 18 | Microcrystalline cellulose Avicel ® pH 102 | 60 |
| 19 | Inositol | 100 |
| 20 | Fumed Silica (Cab-O-Sil) | 25 |
| 21 | Dibasic Calcium Phosphate | 50 |
| 24 | Polyvinylpyrrolidone PVP K-25 | 10 |
| 24 | Methocel ® K4M CR grade | 80 |
| | Ethyl alcohol anhydrous for granulation | |
| 25 | Mg Stearate | 5.3 |
| | TABLET WEIGHT, mg | 731 |

### EXAMPLE 2

### Self-Emusifying Composition Containing Water Insoluble Beta-Sitosterol, Octacosanol, Saw Palmetto Extract, Gugulipid Extract, Tocopherol Acetate and Coenzyme Q10 in Combination with Water Salable Herbal Extracts, Vitamins and Trace Metals

Example 2 is similar to Example 1 and is presented in Table 2. The preparation method and tablet properties are analogous to those described in Example 1.

TABLE 2

| | COMPOUND | PER TABLET, mg |
|---|---|---|
| | Part A | |
| 1 | Hawthorn extract | 50 |
| 2 | Ursolic acid | 10 |
| 3 | Saw palmetto 90% liquid extract | 40 |
| 4 | Gugulipid extract 7.5% | 150 |
| 5 | Coenzyme Q10 | 10 |
| 6 | Dandellion root extract | 100 |
| 7 | Zinc gluconate | 50 |
| 8 | Alpha-Tocopherol acetate | 40 |
| 9 | Folic acid | 0.5 |
| 10 | Octacosanol | 1 |
| 11 | Beta-sitosterol | 20 |
| 12 | TPGS | 20 |
| 13 | Mirj 52 ® | 20 |
| | Ethyl alcohol anhydrous for granulation | |
| 14 | Fumed silica (Cab-O-Sil) | 40 |
| 15 | Dibasic Calcium Phosphate | 60 |
| 16 | Microcrystalline cellulose Avicel ® pH 102 | 50 |
| | Part B | |
| 17 | Fumed silica (Cab-O-Sil) | 25 |
| 18 | Dibasic Calcium Phosphate | 50 |
| 19 | Polyvinylpyrrolidone PVP K-25 | 20 |
| 20 | Methocel ® K4M CR grade | 20 |
| | Ethyl alcohol anhydrous for granulation | |
| 21 | Magnesium Stearate | 5 |
| | TABLET WEIGHT, mg | 781.5 |

### EXAMPLE 3

### Lipobloc Self-Emulsifying Controlled Release Tablet (Wet Granulation Formula) Water Dispersible Beta-Sitosterol and Octacosanol Delivery to Decrease Cholesterol Absorption During the Meals

The composition of Example 3 was prepared as described in Example 1. Table 3 shows the composition highly loaded with sitosterols. After swallowing, the tablet dissolves gradually releasing the tiny emulsion containing dissolved sitosterol. Due to the high level of dispersion and increased surface area, the composition provided the high bioavailability of entrapped phytosterol. This combination is designed for reducing cholesterol absorption.

TABLE 3

| | COMPOUND | PER TABLET, mg |
|---|---|---|
| 1 | Beta-Sitosterol | 50 |
| 2 | Alpha-Tacopherol acetate | 60 |
| 3 | Imwitor ™ 308 | 40 |
| 4 | Octacosanol | 1 |
| 5 | Mirj ® 52 | 50 |
| | Ethyl alcohol anhydrous for granulation | |
| 6 | Fumed silica (Cab-O-Sil) | 40 |
| 7 | Dibasic Calcium Phosphate | 50 |
| 8 | Microcrystalline cellulose Avicel ® pH 102 | 40 |
| 9 | Fumed silica (Cab-O-Sil) | 30 |
| 10 | Dibasic Calcium Phosphate | 20 |
| 11 | Lactose spray dried | 40 |
| 12 | Methocel ® E15 | 40 |
| 13 | Polyvinylpyrrolidone PVP K-25 | 15 |
| | Ethyl alcohol anhydrous for granulation | |
| 14 | Magnesium Stearate | 5 |
| | TABLET WEIGHT, mg | 481 |

Compositions presented in Tables 1-3 were prepared by traditional wet granulation using ethyl alcohol as a solvent for granulation solution preparation. In situations when the use of organic solvents must be limited, a dry mixing followed by forced compaction can be used for granulation process. Table 4 presents formulation of Example 4, related to Example 3, but designed for dry mixing/compaction granulation process.

TABLE 4
LIPOBLOC SELF-EMULSIFYING CONTROLLED RELEASE
TABLET (DRY MIXING/COMPACTION GRANULATION PROCESS)

| | COMPOUND | PER TABLET, mg |
|---|---|---|
| 1 | Beta-Sitosterol | 50 |
| 2 | Alpha-Tocopherol acetate | 60 |
| 3 | Imwitor ™ 308 | 40 |
| 4 | Octacosanol | 1 |
| 5 | Mirj ® 52 | 50 |
| 6 | Fumed silica (Cab-O-Sil) | 50 |
| 7 | Dibasic Calcium Phosphate | 50 |
| 8 | Microcrystalline cellulose Avicelc ® pH 102 | 60 |
| 9 | Fumed silica (Cab-O-Sil) | 30 |
| 10 | Dibasic Calcium Phosphate | 60 |
| 11 | Lactose spray dried | 110 |
| 12 | Polyvinylpyrrolidone PVP K-25 | 15 |
| 13 | Methocel ® E15 | 40 |
| 14 | Polyethylene glycol PEG 8000 (powder) | 20 |
| 15 | Magnesium Stearate | 4 |
| | TABLET WEIGHT, mg | 640 |

### EXAMPLE 4

### Granulation Process for Beta-Sitosterol and Octacosanol Self-Emulsifying Controlled Release Delivery Tablet Formulation

All calculations, were made for 10,000 tablets (theoretical yield).

Using a suitable mixer mix at low speed, 500 g of PEG-40 stearate, 400 g of lmwitor-308 and 600 g of alpha-tocopherol acetate together were added and heated at between 55° and 60° C. until melted. 500 g of beta-sitosterol and 10 g of Octacosanol were added to the melted mixture and slowly stirred until complete dissolution of the components. The temperature of the melted mass was carefully controlled at (75°±5° C.) 500 g of colloidal silicon dioxide (Cab-O-Sil) and 500 g of dibasic calcium phosphate, were mixed separately and passed through a 12-mesh screen and slowly added with continuous mixing to the melted mass.

400 g of microcrystalline cellulose (Avicel™ pH 102) was gradually added and mixed well using the mixer at low speed until a homogenous granulate was formed. To the mixture, 300 g of Cab-O-Sil, 600 g of dibasic calcium phosphate were added and mixed slowly. 400 g of Methocel E-15, 600 g of Lactose (spray dried) and 150 g of Povidone PVP K-25 were subsequently added and mixed for 15 minutes at low speed. The mixture was transferred into a roller compactor and slabs prepared using adequate compacting parameters. Alternatively, intermediary tablets could be prepared as slabs using any suitable tablet press at low speed.

The slabs were ground using a grinder equipped with the stainless steel screen of 16 mesh. 200 g of PEG 8000, 500 g of spray dried lactose, 200 g of microcrystalline cellulose were mixed and passed through the 16 mesh sieve using Frewitt. The milled slabs were combined with the mixture and mixed 5 minutes using an appropriate blender. 40 g of magnesium stearate was passed through a 16 mesh screen and added to the blend and mixed using the blender for 5 minutes. The tablets were pressed (oval or capsule shape tablets, hardness 4-10 kg, tablet weight 640 mg) using a tablet press machine (Manesty™ B3B, Betapress), with precompression.

In respect of the dissolution behaviour, dissolution of the prepared tablets was investigated using USP dissolution apparatus No. 2 (paddles 50 or 100 rpm, 900 ml of simulated gastric fluid without enzymes, temperature 37° C.). All tablets dissolved in a controlled manner, with no burst or dumping release of included components. Dissolution time was adjusted to 2-3 hours, providing optimal delivery of sitosterol with the meals. If necessary, any dissolution behavior can be constructed for the tablet, using different polymers, water soluble excipients and disintegrants, according to the prior art.

During dissolution, the oil phase, entrapped in the matrix, formed an oil-in-water emulsion. Included active components such as sitosterol and octacosanol were released as solution in the oil droplets of this emulsion. Particle size analyses of the formed emulsion showed that a significant part of the oil droplets in the formed OW emulsion had submicron size and the rest droplets had a size below 10 microns.

Biological Observations:

Tablets (examples 2, 3 and 4), consumed in an amount of 2-6 tablets per day (1-3 times before or with meals) by volunteers, showed negligible side effects, significant appetite suppression and visible weight decrease. A 6%-15% decrease in total cholesterol level (Formulation of Example 3) with no additional diet after 2 months treatment was observed.

Although embodiments of the invention have been described above, it is not limited thereto and it will be apparent to those skilled in the art that numerous modifications form part of the present invention insofar as they do not depart from the spirit, nature and scope of the claimed and described invention.

We claim:

1. A composition for bodyweight and cholesterol control, comprising:
at least one phytosterol in an amount between 5% and 50% by weight;
a physiologically or pharmaceutically acceptable surfactant in an amount between 0.1% and 50% by weight;
a lipid phase for containing said phytosterol and said surfactant, said lipid phase present in an amount from 1% and 50% by weight; and
a pharmaceutically acceptable excipient for facilitating absorption of said lipid phase present in an amount from between 10% and 80% by weight.

2. The composition as set forth in claim 1, wherein said composition further includes a physiologically acceptable nutrient in an amount from between 0.005% and 90% by weight.

3. The composition as set forth in claim 2, further including a second excipient for controlling dissolution rate of said composition.

4. The composition as set forth in claim 1, wherein said nutrient is selected from the group consisting of vitamin compounds, minerals, amino acids and plant extracts.

5. The composition as set forth in claim 1, wherein said phytosterol includes the β-sitosterol, sitosternol, campesterol.

6. The composition as set forth in claim 1, wherein said composition is a self-emulsifying composition.

7. The composition as set forth in claim 1, wherein said pharmaceutically acceptable surfactant is present in an amount from between 0.1% and 50% by weight of said composition.

8. The composition as set forth in claim 4, wherein said nutrient is present in an amount from between 0.01% and 50% by weight.

9. A composition for bodyweight and cholesterol control, comprising:
a self-emulsifying base providing in the body submicron particles of dissolve components, said components including:
at least one phytosterol in an amount between 5% and 50% by weight;
a physiologically or pharmaceutically acceptable surfactant in an amount between 0.1% and 50% by weight;
a lipid phase for containing said phytosterol and said surfactant, said lipid phase present in an amount from 1% and 50% by weight; and
a pharmaceutically acceptable excipient for facilitating absorption of said lipid phase present in an amount from between 10% and 80% by weight.

10. The composition as set forth in claim 9, wherein said composition further includes a physiologically acceptable nutrient in an amount from between 0.005% and 90% by weight.

11. The composition as set forth in claim 10, further including a second excipient for controlling dissolution rate of said composition.

12. The composition as set forth in claim 10, wherein said nutrient is selected from the group consisting of vitamin compounds, minerals, amino acids and plant extracts.

13. The composition as set forth in claim 10, wherein said phytosterol includes the β-sitosterol, sitosternol, campesterol.

14. The composition as set forth in claim 10, wherein said pharmaceutically acceptable surfactant is present in an amount from between 0.1% and 50% by weight of said composition.

15. A method of preparing a composition as claimed in claim 1, comprising the steps of:
a) dissolving said at least one phytosterol and said surfactant in said lipid phase;
b) formulating a self-emulsifying mixture by mixing dissolved materials from step a) with a volatile organic solvent and granulation of the obtained solution with nutrient and other excipients;
c) drying said mixture from step b);
d) mixing dried mixture from step c) with granulating components; and
e) granulating said mixture from step d).

16. The method as set forth in claim 15, further including the step of compressing granulated material from step e) into tablets.

17. The method as set forth in claim 15, wherein said step of drying includes drying at 40° C.
